(19) United States
(12) Patent Application Publication
(10) Pub. No.: US 2007/0208080 A1
(43) Pub. Date: Sep. 6, 2007

(54) A COMPOSITION CONTAINING A THIOUREA DERIVATIVE FOR PREVENTING OR TREATING PRURITIC OR IRRITANT SKIN DISEASES

(75) Inventors: Hyun Ju Koh, Gyeonggi-do (KR); Jin Kyu Choi, Gyeonggi-do (KR); Yeon Su Jeong, Gyeonggi-do (KR); Kyung Min Lim, Gyeonggi-do (KR); Joo-Hyun Moh, Gyeonggi-do (KR); Joon Ho Bae, Seoul (KR); Dae Kwon Kim, Seoul (KR); Kwang Mi Kim, Seoul (KR); Chang Hoon Lee, Seoul (KR); Yung Hyup Joo, Seoul (KR); Song Seok Shin, Gyeonggi-do (KR); Young-Ho Park, Seoul (KR); Young Chul Sim, Gyeonggi-do (KR)

Correspondence Address: HESLIN ROTHENBERG FARLEY & MESITI PC, 5 COLUMBIA CIRCLE, ALBANY, NY, 12203, US

(73) Assignee: AMOREPACIFIC CORPORATION, Seoul (KR)

(21) Appl. No.: 11/573,740

(22) PCT Filed: Aug. 19, 2005
(86) PCT No.: PCT/KR05/02735

§ 371 Date: Feb. 15, 2007

(30) Foreign Application Priority Data
Aug. 19, 2004 (KR) 10-2004-0065260

(51) Int. Cl. A61K 31/265; A61K 31/17

(52) U.S. Cl. 514/512; 514/586

(57) ABSTRACT

Disclosed herein is a composition for preventing or treating pruritic or irritant skin diseases which comprises, as a vanilloid receptor antagonist, a thiourea derivative, a pharmaceutically acceptable salt thereof, a hydrate thereof or a solvate thereof, together with a pharmaceutically acceptable carrier.

### TECHNICAL FIELD

The present invention relates to a composition for preventing or treating pruritic or irritant skin diseases which comprises, as a potent antagonist of vanilloid receptor 1 (VR1), a thiourea derivative, a pharmaceutically acceptable salt thereof, a hydrate thereof or a solvate thereof, together with a pharmaceutically acceptable carrier.

### BACKGROUND ART

Most skin diseases, including atopic dermatitis (Wahlgren, 1991, Acta Derm. Venerenol. Suppl., 165, pp 1-53), contact dermatitis (Meding, 1990, Acta Derm. Venerenol. Suppl., 153, pp 1-43), urticaria (Scoter, 1998, Dermatology in general medicine. 5^{th} ed., pp 1409-1419), psoriasis (Krueger et al., 2001, Arch. Dermatol., 137, pp 280-284) and the like, are accompanied with clinical symptoms, such as skin irritation and itching. Particularly, systemic diseases having no relation with the skin, e.g., chronic renal failure (Schwartz et al., 1999, Nephrol. Dial. Transplant., 14, pp 834-839) and biliary atresia (Jones, 1999, Hepatology, 29, pp 1003-1006), also involve pruritus. Scratching the skin is a specific behavioral response to skin irritation and itching, thus causing dermatitis, such as scratches and erythema. Further, there may increase a danger of skin infections. Many inducers directly cause release of neuropeptides from the sensory nerves or promote itching-inducing mediators in mast cells and keratinocytes to induce skin irritation and itching (Yosipovitch et al., 2003, Lancet, 361, pp 690-694).

Although therapeutic agents, including corticosteroids, antihistamines, immunosuppressants, etc., have been used to treat itching, as is widely known, they elicit side effects. Steroids for topical application make the skin thin, cause changes in skin color, or induce eruption. Particularly, the long-term use of large amounts of steroids for topical application may cause systemic side effects, e.g., inhibition of adrenal functions. The first-generation antihistamines are mainly used by systemic administration and show sedative effects due to their antiparasympathetic activity. Chlorpheniramine, a first-generation antihistamine, does not inhibit itching in atopic dermatitis patients upon topical administration (Munday et al., 2002, Dermatology, 205. pp 40-45). The use of topical antihistamines in the treatment of atopic dermatitis is not recommended because of the danger of subcutaneous hypersensitivity. Simultaneous administration of ebastine or terfenadine, second-generation antihistamines free of sedative effects, with a cytochrome P450 activity-inhibiting drug (ketoconazole or erythromycin) may cause arrhythmia (Hey et al., 1996, Arzneimittelforschung, 46, pp 159-163). The immunosuppressant, cyclosporine, may cause serious side effects, such as hypertension, nephrotoxicity and drug interactions, when systemically administered, and shows poor efficacy when topically administered, because its molecular weight is too high to penetrate the skin. Protopic (tacrolimus, FK506) and Elidel (pimecrolimus) are calcinurin inhibitors that have recently been developed as topical preparations. These calcinurin inhibitors cause fewer side effects and are more efficacious than cyclosporine, but they cause side effects, such as burning sensation, itching and erythema, in their initial stage of use (Gupta et al., 2002, JEADV, 16, pp 100-114; Gupta and Chow, 2003, JEADV, 17, pp 493-503). Although the mechanisms are not clearly established, calcinurin inhibitors, such as cyclosporine, tacrolimus, and pimecrolimus increase the influx of calcium into cells in the sensory nerve fibers to release neurotransmitters and degranulate mast cells (Stander and Luger, 2003, Hautarzt, 54, pp 413-417). Similar effects are also observed during capsaicin treatment. Recent investigations report that activity of vanilloid receptor (TRPV1) is controlled by calcineurin in sensory neurons (Wu et al., 2005, J. Biol. Chem., 280, pp 18142-51). This suggests the possibility that the side effects, such as skin irritation and itching, caused by calcinurin inhibitors may be associated with vanilloid receptors. So far few therapeutic agents have been developed to treat skin irritation. Skin irritation is currently treated by the removal of irritants or the use of steroids.

In addition to these treatments, capsaicin creams, doxepin creams, and aspirin are used as topical itching therapeutic agents. Capsaicin creams desensitize pain-transmission nerves to exert antipruritic effects, but cause irritation at the application sites in their initial stage of use and cannot thus be used to treat most inflammatory skin diseases accompanying skin irritation (Wachtel, 1999, Reg. Anesth. Pain Med., 24, pp 361-363). The skin irritation is well known as a characteristic side effect caused by vanilloid receptor agonists, such as capsaicin. It was reported that doxepin, a tricyclic antidepressant, functions in both H1 and H2 histamine receptors to relieve pruritus, but causes side effects, such as burning, stinging and drowsiness, in its initial stage of application (Drake et al., 1994, J. Am. Acad. Dermatol., 31, pp 613-619). Aspirin is effective on pruritus upon topical application, but shows no substantial effect upon oral administration (Daly and Shuster, 1986, Br. Med. J., 293, p 907). Moreover, aspirin causes representative gastrointestinal side effects through the inhibition of cyclooxygenase in view of the mechanism of action.

On the other hand, vanilloid receptor (VR1) present in nerve fibers transmitting harmful stimuli plays a crucial role as an integral transmitter of various endogenous harmful physical/chemical stimuli, such as protons (acids), heat, arachidonic acid derivatives, etc. (Tominaga et al., 1999, Neuron, 21, pp 531-543). In recent years, it has been clearly found that primary afferent sensory nerves containing a vanilloid receptor are distributed not only in most organs of the body, including the digestive organs, the respiratory organs and the bladder, but also in the skin (Stander et al., 2004, Exp. Dermatol. 13, pp 129-139). Activation of the vanilloid receptor by endogenous/exogenous stimuli leads not only to transmission of noxious stimuli, but also to release of neuropeptides, such as substance P, calcitonin gene-related peptides (CGRPs), and the like, thereby causing neurogenic inflammation. It has been reported, surprisingly, in recent scientific papers that vanilloid receptors are distributed in skin epidermal keratinocytes as well as in primary afferent sensory nerves (Denda et al., 2001, Biochem. Biophys. Res. Commun., 285, pp 1250-1252; Inoue et al., 2001, Biochem. Biophys. Res. Commun., 291, pp 124-129), and that when the receptors are activated, inflammatory factors are released (Southall et al., 2003, J. Pharm. Exp. Thera., 304, pp 217-222). That is, vanilloid receptors are present in the sensory nerves of the skin and skin epidermal keratinocytes, and are involved in the transmission of various harmful stimuli, such as skin irritation and itching, and pain, thereby having close correlation with etiology of dermatological diseases and disorders, such as skin inflammation, due to neurogenic/non-neurogenic factors.

Since actions of vanilloid receptor antagonists and agonists in the skin can be explained by blocking of the actions of vanilloid receptors and blocking/inhibition of the actions of primary afferent sensory nerves or keratinocytes containing the vanilloid receptors, it can be said that their application to skin diseases is almost identical.

Animal tests and clinical tests on major vanilloid receptor agonists, including capsaicin, have already been completed or are under way in many directions. These tests mainly employ topical administration routes due to limitations of side effects/toxicity, including irritation, and transdermal absorption. In actuality, vanilloid receptor agonists have been clinically applied to skin diseases, particularly, for the purpose of antipruritic effects, such as psoriasis, pruritus in patients suffering from chronic renal failure undergoing hemodialysis, aquagenic pruritus, pruritus due to vulvar vestibulitis, neurogenic pruritus such as notalgia paraesthetica and brachioradial pruritus, lichen simplex chronicus, and the like. According to the results of the clinical evaluation, the vanilloid receptor agonists, including capsaicin, exhibited therapeutic effects, but were disadvantageously irritating in their initial stage of application (Szallasi and Blumberg, 1999, Pharmacol. Rev., 51, pp 159-211). The vanilloid receptor agonists exert their pharmacological effects while undergoing typical stages consisting of sensitization to nerve cells, desensitization and neurotransmission blocking/neurotoxicity, according to their action mechanism. The actual pharmacological efficacy is achieved through desensitization and functional inhibition of the vanilloid receptors and nerves including the vanilloid receptors. At this time, the early sensitization stage involves unnecessary side effects, such as irritation.

However, since vanilloid receptor antagonists specifically block the functions of vanilloid receptors based on the mechanism of action, they have advantages that neurogenic inflammation and secretion of inflammatory factors in the skin epidermal keratinocytes can be blocked without undergoing an initial sensitization reaction, which is a side effect of the vanilloid receptor agonists. The present invention has significant meaning in that it was proven by specific experiments that vanilloid receptor antagonists exhibit antipruritic effects and inhibitory effects on skin irritation.

According to a recent scientific paper employing the human skin keratinocytes, activation of VR1 elicits inflammation and this kind of expression of various inflammatory cytokines is inhibited by capsazepine, a well-known vanilloid receptor antagonist (Southall et al., 2003, J. Pharm. Exp. Thera., 304, pp 217-222). Further, the applicability of capsazepine to neurogenic skin diseases, such as skin irritation and pruritus, is described in claims of U.S. Pat. No. 6,048,855. However, no specific and detailed experimental results capable of proving the clinical applicability to skin diseases, particularly, antipruritic effects and inhibitory effects on skin irritation have been reported.

Thus, the present inventors have specifically assessed the efficacy of compounds according to the present invention in animal models for itching and skin irritation. Since it is nearly impossible to communicate with experimental animals, itching and skin irritation should be evaluated through the observation of the routine behaviors of the experimental animals. To this end, pruritogen or irritant is intradermally injected into the experimental animals, because intradermal injection can localize the injected substance at the injection site for a relatively long period of time, making it possible to observe the scratching behavior of the injection site (Kuraish et al., 1995, Eur. J. Pharmacol., 275, pp 229-233). In this case, rodents are preferably employed as the experimental animals due to ease of observation and experimentation. The experimental animals exhibit quite a similar behavioral pattern by injection of itching-inducing and irritant substances. In the case where pruritogens are injected, experimental animals commonly lick or bite the injection sites with mouth or scratch the injection sites with forepaws or hind paws. The same behaviors are observed in the case where skin-irritant substances are injected (Green, 2000, Am. J. Contact Dermat., 11, pp 170-180). However, in most hairy animals, including rodents, the behaviors of licking or biting a part of the body with mouth, or scratching a part of the body with forepaw are usually observed without injection of pruritogen or irritant. However, the behaviors of scratching a part of the body with hind paws is rarely observed in routine circumstance, and in the case where an pruitogen or irritant is injected at a site of upper dorsal area near the neck which cannot be reached by forepaw, the behavior of scratching the injection site with hind paws is significantly increased. Therefore, the hind paws scratching directed at a pruritogen or irritant injected site can be considered as an indirect indicator of itching or skin irritation in the experimental animals (Kuraish et al., 1995, Eur. J. Pharmacol., 275, pp 229-233). In this case, however, it is difficult to determine if the injected substance is a pruritogen or irritant. To solve this problem, clinical results obtained from human study are used. It is known that obviously pruritogens in human induce itching in rodents. Examples of such pruritogens include C48/80 (Fjellner et al., 1982, Acta Derm Venereol., 62, pp 137-140), histamines (Maekawa et al., 2000, Jpn. J. Pharmacol., 84, pp 462-466), substance P (Hagemark et al., 1978, J. Invest. Dermatol., 71, pp 233-235), serotonin (Berendsen et al., 1991, Eur. J. Pharmacol., 194, pp 201-208), and PAF (Fjellner et al., 1985, Acta Derm Venereol., 65, pp 409-412). Likewise, it is known that the skin scratching of rodents is induced by irritant in humans. Examples of such irritants include erosive organic solvents (JP2001-321016), hypotonic solutions having an osmotic pressure lower than that of body fluids (Hwang et al., 1986, Life Sci., 30, pp 2389-2396), pyrethroid (Gargen et al., 1984, Toxicol. Appl. Pharmacol., 76, pp 270-279), tacrolimus (Fuchs et al., 2002, Contact Dermatitis, 45, pp 290-294), and retinoic acid (Varani et al., 2003, Arch. Dermatol. Res., 295, pp 255-262). Alternatively, pruritogens and irritants can be empirically determined. Pruritogens increase the scratching behavior in a dose-dependent manner, but substantial dose dependence is not observed in most irritants. In addition, it is known that the frequency of the scratching behavior induced by pruritogens is generally higher than that induced by irritants (Jinks et al., 2002, J. Neurophysiol., 87, pp 1280-1289). Therefore, the present inventors utilized the scratching behavior induced by C48/80 and histamine as an index of itching, and the scratching behavior induced by tacrolimus as an index of skin irritation in animal models. In addition, the present inventors utilized the occurrence of erythema induced by retinoic acid as an index of direct skin irritation.

### DISCLOSURE OF THE INVENTION

### Technical Problem

Based on the theoretical background discussed above, an object of the present invention is to provide a composition for preventing or treating pruritic or irritant skin diseases which comprises a thiourea derivative as a vanilloid receptor antagonist, a pharmaceutically acceptable salt thereof, a hydrate thereof or a solvate thereof, in combination with a pharmaceutically acceptable carrier.

### Technical Solution

In accordance with an aspect of the present invention, there is provided a composition for preventing or treating pruritic or irritant skin diseases wherein the composition comprises a thiourea derivative as a vanilloid receptor antagonist represented by Formula (1), a pharmaceutically acceptable salt thereof, a hydrate thereof, or a solvate thereof, in combination with a pharmaceutically acceptable carrier;

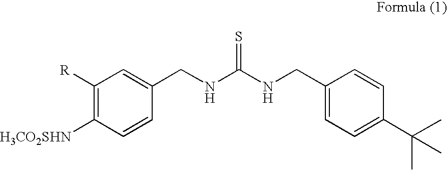

wherein R is hydrogen, C_{1˜5} alkyl, C_{2˜5} alkenyl, C_{1˜5} alkoxy, hydroxyl, halogen, nitro, cyano, methoxycarbonyl or carboxyl.

The substitutent R in Formula (1) is preferably hydrogen, methyl, ethyl, propyl, vinyl, propenyl, methoxy, ethoxy, propoxy, hydroxyl, fluoro, chloro, bromo, iodo, nitro, cyano, methoxycarbonyl or carboxyl.

Particularly preferred compounds that can be represented by Formula (1) include:

1-(4-t-butylbenzyl)-3-(3-fluoro-4-methanesulfonylaminobenzyl)thiourea (compound 1);

1-(4-t-butylbenzyl)-3-(3-chloro-4-methanesulfonylaminobenzyl)thiourea (compound 2);

1-(4-t-butylbenzyl)-3-(3-methyl-4-methanesulfonylaminobenzyl)thiourea (compound 3);
1-(4-t-butylbenzyl)-3-(4-methanesulfonylaminobenzyl)thiourea (compound 4); and
1-(4-t-butylbenzyl)-3-(3-vinyl-4-methanesulfonylaminobenzyl)thiourea (compound 5).

The thiourea derivatives of Formula (1) were developed as selective antagonists of vanilloid receptors by the present inventors, most of which are disclosed in PCT publication WO 02/116318.

Salts of the thiourea derivatives of Formula (1) include salts with inorganic acids, e.g., hydrochloric acid, hydrobromic acid, hydroiodic acid, sulfuric acid, nitric acid, phosphoric acid, and perchloric acid; salts with organic acids, e.g., methanesulfonic acid, trifluoromethanesulfonic acid, benzenesulfonic acid, p-toluenesulfonic acid, acetic acid, trifluoroacetic acid, propionic acid, tartaric acid, fumaric acid, maleic acid, malic acid, oxalic acid, succinic acid, citric acid, benzoic acid, mandelic acid, cinnamic acid, lacetic acid, glycolic acid, glucuronic acid, ascorbic acid, nicotinic acid, and salicylic acid; and salts with acidic amino acids, e.g., aspartic acid and glutamic acid. In addition, included are ammonium salts; salts with amines, e.g., methylamine, dimethylamine, trimethylamine, dicyclohexylamine, tris(hydroxymethyl)aminomethane, N,N-bis(hydroxyethyl)piperazine, 2-amino-2-methyl-1-propanol, ethanolamine, N-methylglucamine, and L-glucamine; and salts with basic amino acids, e.g., lysine, δ-hydroxylysine, and arginine.

The compounds of Formula (1) and salts thereof may exist in the form of hydrates and solvates.

The pharmaceutical composition of the present invention can be administered via various routes, e.g., orally, parenterally, subcutaneously, and intradermally. Intradermal administration and topical application are more preferred. The preferable dose level of the compounds according to the present invention depends upon a variety of factors including the condition and body weight of the patient, severity of the particular disease, dosage form, and route and period of administration, but may be appropriately chosen by those skilled in the art. The compounds of Formula (1) are preferably administered in amounts ranging from 0.001 to 100 mg/kg of body weight per day, and more preferably from 0.01 to 30 mg/kg of body weight per day. Doses may be administered once a day, or several times a day in divided portions. The compounds of Formula (1) are used in an amount of 0.0001˜10% by weight, based on the total amount of the composition of the present invention.

The composition of the present invention is generally administered in the form of a pharmaceutical preparation, which is prepared by mixing the ingredient component with a pharmaceutically acceptable carrier or a diluent. Examples of suitable pharmaceutical preparations include powders, tablets, capsules and liquids for oral administration; transdermal absorption preparations; emulsions; suspensions; patches, creams, and cataplasma for external application; intravenous injectable preparations; and intramuscular injectable preparations. The pharmaceutical composition can be formulated by common techniques. Of these, external preparations are particularly preferred. Specific examples of external preparations include, but are not limited to, creams, ointments, gels, emulsions, sticks, packs, and solutions in organic solvents.

Suitable pharmaceutically acceptable carriers and diluents include those commonly used in the pharmaceutical field, which do not react with the compounds used in the present invention. Suitable pharmaceutically acceptable carriers and diluents for the production of powders, tablets, capsules and the like include: excipients, such as corn starch, lactose, mannitol, and microcrystalline cellulose; disintegrants, such as croscarmellose sodium, potato starch, and white refined sugar; binders, such as refined gelatin, arabic gum, methylcellulose, ethylcellulose and povidone; and lubricants, such as magnesium stearate, hard anhydrous silicic acid, and talc.

Tablets can be coated with coating agents by common techniques. Suitable coating agents include hydroxypropylmethylcellulose, hydroxypropylmethylcellulose phthalate, cellulose acetate phthalate, titanium oxide, polysorbates, and white refined sugar.

Suitable ingredients used to produce skin external preparations include liquid oils and fats, solid oils and fats, white waxes, hydrocarbons, higher fatty acids, higher alcohols, esters, surfactants, moisturizing agents, water-soluble polymeric compounds, thickeners, coating agents, lower alcohols, polyhydric alcohols, saccharides, amino acids, organic amines, pH-adjusting agents, antioxidants, fragrances, and water. If needed, these ingredients may be appropriately blended with one another.

Examples of suitable adhesive bases for patches include polymeric bases, such as acrylic copolymers, polyvinylpyrrolidone and polyisobutylene; and plasticizers, such as triethyl citrate, triethylacetyl citrate, glycerin, propylene glycol, and polyethylene glycol.

Injectable preparations may be formulated by dissolving salts of the compound of Formula (1) in distilled water. If necessary, additives, such as isotonic agents, analgesic agents, pH-adjusting agents, dissolution assistants, buffering agents and preservatives, can be further added. Injectable preparations may be a suspension produced by suspending the compounds of Formula (1) in distilled water for injection or vegetable oil. If necessary, additives, such as bases and suspending agents, can be further added. In addition, injectable preparations may take the form of powders or may be in lyophilized forms. These dosage forms are dissolved before use, and excipients may be further added thereto.

Generally, pharmaceutical additives can be mixed in amounts ranging from 1% to 90% by weight, based on the weight of active ingredients.

The present invention also provides a method for preventing or treating pruritic or irritant skin diseases, comprising administering to a mammal in need thereof a therapeutically effective amount of the composition of the present invention.

The composition of the present invention is used to prevent or treat pruritic or irritant skin diseases.

The composition of the present invention is used in the preparation of medicaments for the prevention or treatment of pruritic or irritant skin diseases.

### MODE FOR CARRYING OUT THE INVENTION

The present invention will now be described in more detail with reference to the following experimental examples and formulation examples. However, these examples are given for the purpose of illustration and are not to be construed as limiting the scope of the invention.

### EXPERIMENTAL EXAMPLE

### Evaluation of Biological Efficacy

It was confirmed that the thiourea derivatives of the present invention as vanilloid receptor antagonists exerted antipruritic effects and inhibitory effects on skin irritation in various animal models. The compounds 1 to 3 were used as preferred thiourea derivatives in the following experimental examples.

### Experimental Example 1

Itching induced by compound 48/80 (C48/80) in ICR mice (Intradermal Administration)

ICR mice suffer from itching upon administration of histamines, and C48/80 (condensation product of N-methyl-p-methoxyphenethylamine with formaldehyde, Sigma, U.S.A.) is a substance that stimulates connective tissues and skin mast cells to release mediators, such as histamines, causing itching. C48/80 causes itching when intradermally administered to humans (Rukwied et al., 2000, Br. J. Pharmacol., 142, pp 114-1120). In this experiment, ICR mice received C48/80 to induce itching. One day before the experiment, the rostral part of the skin on the back of male ICR mice (28˜32 g, 4˜11 mice/group) was clipped. After the drug (compound 1, 50 μl/mouse) was dissolved in hydroxypropyl-β-cyclodextrin (HP-β-CD; Mitsubishi Ltd., Japan) and C48/80 (50 μg/50 μl/mouse) was dissolved in a physiological saline solution, the solutions were simultaneously injected intradermally into the back skin of mice. Immediately after the injection, the mice were placed in a transparent cage and then the frequency of scratching of the injected site by the hind paws was counted for 60 minutes. The frequency of scratching in test groups administered with the drug was compared to that in a control group administered with C48/80 alone. The decrease in the frequency of scratching in the test groups was determined as an index of inhibitory effects on itching. The decrease in the frequency of scratching was expressed as a percentage of inhibition to determine inhibitory effects of each compound on itching. As can be seen from the data shown in Table 1 below, the compound 1 of the present invention exhibited potent antipruritic effects in a dose-dependent manner. In contrast, the first-generation antihistamine, chlorpheniramine, exhibited no inhibitory effects on itching.

TABLE 1

| Dose (mg/mouse, intradermal administration) | % Inhibition (mean ± SE) |
|---|---|
| Compound 1 (0.03) | −6.51 ± 17.39 |
| Compound 1 (0.1) | 36.96 ± 13.44 |
| Compound 1 (0.3) | 44.11 ± 10.36 |
| Compound 1 (1) | 69.03 ± 7.76 |
| chlorpheniramine (0.3) | −77.61 ± 70.54 |

### Experimental Example 2

Itching Induced by Compound 48/80 (C48/80) in BALB/C Mice (Oral administration)

Itching induced by C48/80 in atopic dermatitis patients is not inhibited by histamine (H1) receptor antagonists, which indicates that a substance other than histamine is involved in the itching induction in the atopic patients (Wahlgren et al., 1991, Acta Derm.-Venereol., Suppl. 165, pp 1-53). The frequency of scratching induced by an itching-inducing substance varies depending on mouse strain. For example, it was reported that BALB/C mice are less sensitive to histamines and serotonin than ICR mice (Inagaki et al., 2001, Skin Pharmacol. Appl. Skin Physiol., 14, pp 87-96). This suggests that itching by C48/80 in BALB/C mice is similar to that in atopic dermatitis patients. In this experiment, BALB/C mice received C48/80 to induce itching. One day before the experiment, the rostral part of the skin on the back of BALB/C mice (19˜21 g, 6˜12 mice/group) was clipped. After compound 1 was dissolved in HP-β-CD, and compounds 2 and 3 were dissolved in a physiological saline solution containing 10% ethanol and 10% Tween 80, the solutions were orally administered (10 ml/kg) to the mice. One hour after the administration, C48/80 (50 μg/50 μl/mouse) was dissolved in a physiological saline solution and injected intradermally into the back skin of mice. Immediately after the injection, the mice were housed in a transparent cage and then the frequency of scratching of the injected site by the hind paws was counted for 30 minutes. The frequency of scratching in test groups administered with the drugs was compared to that in a control group administered with C48/80 alone. The decrease in the frequency of scratching in the test groups was determined as an index of inhibitory effects on itching. The decrease in the frequency of scratching was expressed as a percentage of inhibition to determine inhibitory effects of each compound on itching. As shown in Tables 2, 3 and 4 below, the compounds 1 to 3 of the present invention exhibited antipruritic effects in a dose-dependent manner. In contrast, the first-generation antihistamine, chlorpheniramine, and the second-generation antihistamine free of sedative effects, ketotifen, exhibited no inhibitory effects on itching.

TABLE 2

| Dose (mg/mouse, oral administration) | % Inhibition (mean ± SE) |
|---|---|
| Compound 1 (3) | −22.90 ± 13.19 |
| Compound 1 (10) | 42.24 ± 24.48 |
| Compound 1 (30) | 42.97 ± 7.13 |
| Compound 1 (100) | 54.11 ± 18.50 |
| Chlorpheniramine (3) | −22.90 ± 28.21 |
| Ketotifen (10) | −24.91 ± 15.09 |

TABLE 3

| Dose (mg/mouse, oral administration) | % Inhibition (mean ± SE) |
|---|---|
| Compound 2 (3) | 0.89 ± 7.23 |
| Compound 2 (10) | 7.42 ± 10.43 |
| Compound 2 (30) | 44.53 ± 11.07 |

TABLE 4

| Dose (mg/mouse, oral administration) | % Inhibition (mean ± SE) |
|---|---|
| Compound 3 (3) | 8.21 ± 5.82 |
| Compound 3 (10) | 11.78 ± 15.26 |
| Compound 3 (30) | 38.76 ± 7.77 |

### Experimental Example 3

Itching Induced by Histamine in ICR Mice (Oral Administration)

It is known that the itching response induced by histamine in rodents is generally weaker than that in humans (Kuraishi et al., 1995, Eur. J. Pharmacol. 275, pp 229-233). However, it is known that uniquely, ICR mice sensitively respond to histamine, and this leads to itching response (Maekawa et al., 2000, Jpn. J. Pharmacol. 84, pp 462-466). As is widely known, histamines are representative itching mediators and obviously induce itching in human subjects (Simone et al., 1987, Somatosens. Res. 5, pp 81-92). In this experiment, ICR mice received to elicit itching. One day before the experiment, the rostral part of the skin on the back of male ICR mice (28˜32 g, 5˜8 mice/group was clipped. The mice were fasted for 18 hours before experimentation. The drug (compound 1, 0.5 ml/mouse) was dissolved in hydroxypropyl-β-cyclodextrin (HP-β-CD; Mitsubishi Ltd., Japan), and then orally administered to the mice. One hour after the administration, histamine (100 nmol/50 μl/mouse) was dissolved in a physiological saline solution and injected intradermally into the back skin of mice. Immediately after the injection, the mice were placed in a transparent cage, and then the frequency of scratching of the injected site by the hind paws was counted for 30 minutes. The frequency of scratching in test groups administered with the drug was compared to that in a control group administered with histamine alone. The decrease in the frequency of scratching in the test groups was determined as an index of inhibitory effects on itching. The decrease in the frequency of scratching was expressed as a percentage of inhibition to determine inhibitory effects of each compound on itching. From the results shown in Table 5 below, it can be confirmed that the compound 1 of the present invention exhibits potent anti-pruritic effects in a dose-dependent manner. Azelastine, which is known to show superior effects on pruritic diseases through triple activity, i.e., antihistamine activity, inhibition of eosinophil infiltration, and inhibition of mast cells degranulation, showed potent antipruritic effects in a dose-dependent manner.

TABLE 5

| Dose (mg/mouse, oral administration) | % Inhibition (mean ± SE) |
|---|---|
| Compound 1 (10) | 39.8 ± 18.1 |
| Compound 1 (30) | 46.7 ± 24.0 |
| Compound 1 (100) | 75.8 ± 26.9 |
| Azelastine chloride (1) | 63.7 ± 26.6 |
| Azelastine chloride (10) | 91.7 ± 6.8 |

### Experimental Example 4

Inhibition of Skin Irritation Induced by Tacrolimus in ICR Mice (Subcutaneous Administration)

Tacrolimus is an immunosuppressant widely used in liver and renal transplantation (Lawrence, 1998, Dermatol. Ther., 5, pp 74-84). Tacrolimus is currently used in the form of ointment for treating skin diseases, such as atopic dermatitis (Wollenberg et al., 2001, J. Allergy. Clin. Immunol., 107, pp 519-525), psoriasis (Nasr, 2000, Clin. Exp. Dermatol., 25, pp 250-254), allergic contact dermatitis (Lauerma et al., 1992, Lancet, 340, p 556), and the like. However, it was reported that tacrolimus enhances irritation in human models with irritant contact dermatitis (Fuchs et al., 2002, Contact dermatitis, 46, pp 290-294). It was reported that tacrolimus causes side effects, such as skin irritation, itching and erythema, in its initial stage of use (Gupta et al., 2002, JEADV, 16, pp 100-114; Gupta and Chow, 2003, JEADV, 17, pp 493-503). No experimental model of skin irritation by tacrolimus in animal experiments is currently known. Accordingly, tacrolimus as a skin-irritant substance was directly injected to ICR mice by intradermal injection, and then scratching behavior of the mice was observed. One day before the experiment, the rostral part of the skin on the back male ICR mice (30˜33 g, 10 mice/group) was clipped. After the drug (compound 1, 50 μl/mouse) was dissolved in 28% hydroxypropyl-β-cyclodextrin (HP-β-CD; Mitsubishi Ltd., Japan) and tacrolimus (50 μg/50 μl/mouse, Cipla, India) was dissolved in a physiological saline solution containing 5% ethanol and 5% Tween 80, the solutions were simultaneously injected intradermally into the back skin of mice. Immediately after the injection, the mice were placed in a transparent cage and then the frequency of scratching of the injected site by the hind paws was counted for 30 minutes. The frequency of scratching in test groups administered with the drug was compared to that in a group administered with tacrolimus alone. The decrease in the frequency of scratching in the test groups was determined as an index of inhibitory effects on skin irritation. The decrease in the frequency of scratching was expressed as a percentage of inhibition to determine inhibitory effects of each compound on skin irritation. The group administered with tacrolimus exhibited significantly increased skin irritation when compared to the groups administered with the control substance. As can be seen from the data shown in Table 6 below, the compound 1 of the present invention exhibited inhibitory effects on skin irritation in a dose-dependent manner.

TABLE 6

| Dose (mg/mouse, intradermal administration) | % Inhibition (mean ± SE) |
|---|---|
| Compound 1 (0.1) | 33.74 ± 37.73 |
| Compound 1 (0.3) | 52.03 ± 24.14 |
| Compound 1 (1) | 106.91 ± 30.13 |

### Experimental Example 5

Inhibition of Skin Irritation Induced by Retinoic Acid in New Zealand White Rabbits (Topical Administration)

Retinoic acid is widely used as a material for cosmetics and medicines due to its superior skin efficacy, promotion of skin keratinocyte differentiation, acne treatment, reduction of wrinkles and the like (Fisher et al., 1998, J. Investig. Dermatol., 3, pp 61-68). However, it is known that retinoic acid causes irritation in its initial stage of use and further causes side effects, such as skin erythema and edema, when topically applied to the skin (Varani et al., 2003, Arch. Dermatol. Res., 295, pp 255-262). A number of attempts to reduce the skin irritation induced by retinoic acid have been made in many directions (Kim et al., 2003, Toxicol. Letters., 146, pp 65-73). New Zealand white rabbits are widely used in skin irritation experiments because they show superior response to various irritant and there is a small difference between individuals. In this experiment, New Zealand white rabbits received retinoic acid to cause skin irritation. One day before the experiment, hairs around the back of four male New Zealand white rabbits (2.0˜2.5 kg) were shaved. Four application sites, which are symmetrical by two (i.e., right upper site-left upper site and right lower site-left lower site), were marked at the shaved back of each rabbit. 100 μl of 0.025% retinoic acid (Sigma, U.S.A.) was topically applied to all four-application sites. 30 minutes after the application, the two sites marked at the right were treated with the drug (compound 1, 1%/100 μl/each application site), and the two sites marked at the left were treated with a solvent (100 μl/each application site) alone. At this time, retinoic acid and the drug were dissolved in a mixed solution of PEG 400 and ethanol (7:3), and then topically applied twice daily for four consecutive days. On the fifth day, the application sites were visually observed to evaluate the degree of skin irritation. The degree of skin irritation was expressed in a cumulative irritation index. Based on the cumulative irritation index of the control group treated with retinoic acid (0.025%/100 μl/application site) and the solvents alone, the decrease in the cumulative irritation index of the test group treated with both retinoic acid and the drug was expressed as a percentage of inhibition to determine an index of inhibitory effects on skin irritation. The cumulative irritation index was judged based on occurrence of erythema, and scored in accordance with the following criteria: Occurrence of no erythema (0), occurrence of slightly visible erythema (1), occurrence of distinct erythema (2), occurrence of distinct scarlet erythema (3), and occurrence of distinct crimson erythema and scaling (4). As is evident from Table 7, the compound 1 of the present invention exhibited inhibitory effects on skin irritation.

TABLE 7

| | Cumulative | |
|---|---|---|
| | irritation | |
| Dose | index | % Inhibition |
| (100 μl/application site) | (mean ± SE) | (mean) |
| Retinoic acid (0.025%) + solvent | 3.13 ± 0.68 | 0% |
| Retinoic acid (0.025%) + compound 1 (1%) | 1.88 ± 0.45 | 40% |

As apparent from the experimental results, it has been confirmed that the compounds of Formula 1, pharmaceutically acceptable salts thereof, hydrates thereof and solvates thereof according to the present invention exhibit antipruritic effects and inhibitory effects on skin irritation.

### Formulation Example 1

Compound 1, 2 or 3 was dissolved in a solvent and then the other ingredients were added thereto in accordance with the composition indicated in Table 8 below. The resulting mixture was homogenized to formulate a preparation for external application.

TABLE 8

| Ingredients | Content (g/preparation) |
|---|---|
| Compound 1, 2 or 3 | 1 |
| Ethanol | 10 |
| Poloxamer 407 | 20 |
| Oleic acid | 1 |
| Purified water | 50 |

### Formulation Example 2

A preparation having the composition indicated in Table 9 below was formulated in the same manner as in Formulation Example 1.

TABLE 9

| Ingredients | Content (g/preparation) |
|---|---|
| Compound 1, 2 or 3 | 1 |
| Ethanol | 10 |
| Carbopol | 2 |
| Oleic acid | 1 |
| Purified water | 50 |

### Formulation Example 3

A preparation having the composition indicated in Table 10 below was formulated in the same manner as in Formulation Example 1.

TABLE 10

| Ingredients | Content (g/preparation) |
|---|---|
| Compound 1, 2 or 3 | 1 |
| Isopropyl myristate | 40 |
| Liquid Vaseline | 1 |
| Aerosil | 1 |

### Formulation Example 4

A preparation having the composition indicated in Table 11 below was formulated in the same manner as in Formulation Example 1.

TABLE 11

| Ingredients | Content (g/preparation) |
|---|---|
| Compound 1, 2 or 3 | 1 |
| Polyethylene glycol 400 | 50 |
| Ethanol | 30 |

### Formulation Example 5

A preparation having the composition indicated in Table 12 below was formulated in the same manner as in Formulation Example 1.

TABLE 12

| Ingredients | Content (g/preparation) |
|---|---|
| Compound 1, 2 or 3 | 1 |
| Cetyl alcohol | 10 |
| Glyceryl monostearate | 6 |
| PEG-50 stearate | 6 |
| Propylene glycol | 4 |
| Methyl-p-hydroxybenzoate | 0.1 |
| Propyl-p-hydroxybenzoate | 0.1 |
| Purified water | 80 |

### Formulation Example 6

A preparation having the composition indicated in Table 13 below was formulated in the same manner as in Formulation Example 1.

TABLE 13

| Ingredients | Content (g/preparation) |
|---|---|
| Compound 1, 2 or 3 | 1 |
| Ethanol | 40 |
| Sodium polyacrylate | 10 |
| Glycerin | 25 |

### Formulation Example 7

A preparation having the composition indicated in Table 14 below was formulated in the same manner as in Formulation Example 1.

TABLE 14

| Ingredients | Content (g/preparation) |
|---|---|
| Compound 1, 2 or 3 | 1 |
| Ethanol | 40 |
| Carboxymethylcellulose sodium | 0.2 |
| Tween 80 | 0.5 |
| Glycerin | 10 |
| Propylene glycol | 20 |
| Menthol oil | 0.25 |

### Formulation Example 8

A preparation having the composition indicated in Table 15 below was formulated in the same manner as in Formulation Example 1.

TABLE 15

| Ingredients | Content (g/preparation) |
|---|---|
| Compound 1, 2 or 3 | 1 |
| Ethanol | 40 |
| Dimethicone copolyol crosspolymer | 1 |
| dimethicone | |
| Cyclomethicone | 0.5 |
| Dimethicone | 0.6 |
| Propylene glycol | 1 |
| Glycerin | 0.5 |
| Poloxamer 407 | 0.5 |

### Formulation Example 9

TABLE 16

| Ingredients | Content (g/200 g) |
|---|---|
| Compound 1, 2 or 3 | 100 |
| Lactose | 84 |
| Hydroxypropylcellulose (HPC) | 6 |
| Potato starch | 6 |
| Magnesium stearate | 1 |
| Talc | 3 |

A preparation having the composition indicated in Table 16 was formulated in accordance with the following procedure. First, compound 1, 2 or 3 was mixed with lactose and then a binder solution of hydroxypropylcellulose (6 g) in ethanol (40 mL) was added thereto. The mixture was kneaded, passed through a #14 sieve, dried, and passed through a #18 sieve to obtain particles with uniform size. To the particles were added magnesium stearate, talc and potato starch. The resulting mixture was homogenized, and compressed into tablets.

### Formulation Example 10

TABLE 17

| Ingredients | Content | |
|---|---|---|
| Compound 1, 2 or 3 | 2 | mg |
| Polyethylene glycol (M.W. 4,000) | 0.3 | g |
| Sodium chloride | 0.9 | g |
| Polyoxyethylene sorbitan monooleate | 0.4 | g |
| Sodium metabisulfite | 0.1 | g |
| Methyl paraben | 0.18 | g |
| Propyl paraben | 0.02 | g |
| Distilled water for injection | q.s. | |
| Total | 100 | g |

The ingredients indicated in Table 17 were dissolved in distilled water with stirring. The resulting solution was passed through a sterilized 0.2 μm filter, put in a vial, lyophilized, and sealed with a lid to prepare an injectable preparation.

### INDUSTRIAL APPLICABILITY

The composition of the present invention can be used to prevent or treat pruritic or irritant skin diseases.

1. A composition for preventing or treating pruritic or irritant skin diseases wherein the composition comprises a thiourea derivative represented by Formula (1), a pharmaceutically acceptable salt thereof, a hydrate thereof, or a solvate thereof, in combination with a pharmaceutically acceptable carrier; 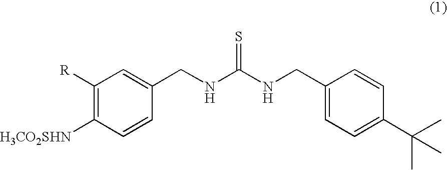
wherein R is hydrogen, C_{1˜5} alkyl, C_{2˜5} alkenyl, C_{1˜5} alkoxy, hydroxyl, halogen, nitro, cyano, methoxycarbonyl or carboxyl.

2. The composition according to claim 1, wherein the substitutent R in Formula (1) is hydrogen, methyl, ethyl, propyl, vinyl, propenyl, methoxy, ethoxy, propoxy, hydroxyl, fluoro, chloro, bromo, iodo, nitro, cyano, methoxycarbonyl or carboxyl.

3. The composition according to claim 1, wherein the thiourea derivative of Formula (1) is:
1-(4-t-butylbenzyl)-3-(3-fluoro-4-methanesulfonylaminobenzyl)thiourea;
1-(4-t-butylbenzyl)-3-(3-chloro-4-methanesulfonylaminobenzyl)thiourea;
1-(4-t-butylbenzyl)-3-(3-methyl-4-methanesulfonylaminobenzyl)thiourea;
1-(4-t-butylbenzyl)-3-(4-methanesulfonylaminobenzyl)thiourea; or
1-(4-t-butylbenzyl)-3-(3-vinyl-4-methanesulfonylaminobenzyl)thiourea.

4. The composition according to claim 1, wherein the composition is formulated into a preparation for oral administration, transdermal administration, topical application, or injection.

5. A method for preventing or treating pruritic or irritant skin diseases wherein the method comprises administering to a mammal in need thereof a therapeutically effective amount of the composition according to claim 1.

6-7. (canceled)
